(19) United States
(12) Patent Application Publication
(10) Pub. No.: US 2024/0008749 A1
(43) Pub. Date: Jan. 11, 2024

(54) HEMODYNAMIC MONITOR WITH NOCICEPTION PREDICTION AND DETECTION

(71) Applicant: EDWARDS LIFESCIENCES CORPORATION, Irvine, CA (US)

(72) Inventors: Christine Lee, Long Beach, CA (US); Feras Al Hatib, Irvine, CA (US); Cristhian M. Potes Blandon, Rancho Santa Margarita, CA (US); Kevin James Moses, Huntington Beach, CA (US); Catherine M. Szyman, Westlake Village, CA (US)

(21) Appl. No.: 18/473,090

(22) Filed: Sep. 22, 2023

Related U.S. Application Data
(63) Continuation of application No. PCT/US2022/020988, filed on Mar. 18, 2022.
(60) Provisional application No. 63/165,702, filed on Mar. 24, 2021.

(51) Int. Cl. A61B 5/02; A61B 5/021; A61B 5/00; A61B 5/349; A61B 5/024

(52) U.S. Cl. CPC A61B 5/02028; A61B 5/02141; A61B 5/7435; A61B 5/7275; A61B 5/4845; A61B 5/349; A61B 5/02405

(57) ABSTRACT

A hemodynamic monitor for detecting nociception of a patient includes a non-invasive blood pressure sensor with an inflatable blood pressure bladder, a pressure controller pneumatically connected to the inflatable blood pressure bladder, and an optical transmitter and an optical receiver that are electrically connected to the pressure controller. The hemodynamic monitor also includes an integrated hardware unit with a system processor, a system memory, and a display with a user interface.

### RELATED APPLICATIONS

This application claims priority to the PCT application having International Application No. PCT/US2022/020988, filed Mar. 18, 2022, and entitled “HEMODYNAMIC MONITOR WITH NOCICEPTION PREDICTION AND DETECTION.” The above-identified PCT application in turn claims priority to U.S. Provisional Patent Application Ser. No. 63/165,702, filed Mar. 24, 2021 and entitled “HEMODYNAMIC MONITOR WITH NOCICEPTION PREDICTION AND DETECTION.” The disclosures of the above patent applications are hereby incorporated by reference in their entirety.

### BACKGROUND

The present disclosure relates generally to hemodynamic monitoring, and in particular to detecting and predicting nociception in a patient using monitored hemodynamic data.

Nociception is the process in which nerve endings called nociceptors detect noxious stimuli and send a signal to the central nervous system which is interpreted as pain. Nociception can cause automatic responses without reaching consciousness or before reaching consciousness, thus an unconscious patient in surgery can experience pain. To prevent a patient from awaking out of surgery in pain, medical workers administer analgesics to the patient before and/or during surgery. However, knowing the amount of analgesic to administer can be difficult as pain thresholds and tolerances vary from patient to patient and the patient is unable to verbally communicate or signal feedback while unconscious. Administering too little analgesic to the patient during surgery can result in the patient awaking in pain after the surgery. Administering too much analgesic to the patient during surgery can result in the patient experiencing nausea, drowsiness, impaired thinking skills, and impaired function.

In view of the negative consequences of administering too little analgesic to the patient and the negative consequences of administering too much analgesic to the patient, a solution is needed that will allow medical workers the ability to detect or predict nociception of an unconscious patient during surgery. Accurately detecting or predicting nociception of a patient during surgery can help medical workers know the appropriate amount of analgesic to administer to the patient so that the patient does not awake from surgery with significant pain, and without providing too much analgesic to the patient.

### SUMMARY

In one example, a hemodynamic monitor is disclosed for detecting nociception of a patient. The hemodynamic monitor includes a non-invasive blood pressure sensor with an inflatable blood pressure bladder, a pressure controller pneumatically connected to the inflatable blood pressure bladder, and an optical transmitter and an optical receiver that are electrically connected to the pressure controller. The hemodynamic monitor also includes an integrated hardware unit with a system processor, a system memory, and a display with a user interface. The system memory includes nociception detection instructions that, when executed by the system processor, are configured to: adjust, by the pressure controller, a pressure within the inflatable blood pressure bladder to maintain a constant volume of an artery of a patient for a period of time based on a feedback signal generated by the optical transmitter and the optical receiver; generate an arterial pressure waveform data of the patient based on the adjusted pressure within the inflatable blood pressure bladder over the period of time; extract a plurality of signal measures from the arterial pressure waveform data of the patient; extract detection input features from the plurality of signal measures that are indicative of a current nociception event of the patient; determine a nociception score of the patient based on the detection input features; generate a first sensory alarm signal configured to generate a first sensory alert that indicates that the patient is experiencing the current nociception event when the nociception score satisfies a predetermined detection criterion; transmit the first sensory alarm signal to the user interface; and output the first sensory alert through the user interface.

In another example, a hemodynamic monitor is disclosed for detecting nociception of a patient. The hemodynamic monitor includes an arterial blood pressure sensor with a housing, a fluid input port connected via tubing to a fluid source, and a catheter-side fluid port connected to a catheter for insertion within an arterial system of the patient. The arterial blood pressure sensor further includes a pressure transducer in communication with the fluid source through the fluid port and an I/O cable in electrical communication with the pressure transducer. The hemodynamic monitor further includes an integrated hardware unit with a system processor, a system memory, a display with a user interface, and an analog-to-digital (ADC) converter. The system memory includes instructions that, when executed by the system processor, are configured to: receive an electrical signal from the pressure transducer over a period of time, the electrical signal based on a pressure of the arterial system of the patient transmitted through the fluid source; convert the electrical signal to a digital signal; generate an arterial pressure waveform data of the patient based on the digital signal; extract a plurality of signal measures from the arterial pressure waveform data of the patient; extract detection input features from the plurality of signal measures that are indicative of a nociception event of the patient; determine a nociception score of the patient based on the detection input features; generate a sensory alarm signal configured to generate a sensory alert that indicates that the patient is experiencing a current nociception event when the nociception score satisfies a predetermined detection criterion; transmit the sensory alarm signal to the user interface; and output the sensory alert through the user interface.

In another example, a method is disclosed for monitoring of arterial pressure of a patient for current or predicted future nociception of the patient. The method includes receiving, by a hemodynamic monitor, a continuous signal from a blood pressure sensor connected to the patient over a period of time. The hemodynamic monitor generates continuously over the period of time an arterial pressure waveform of the patient based on the signal. The hemodynamic monitor extracts a plurality of signal measures from the arterial pressure waveform of the patient. Input features are extracted by the hemodynamic monitor from the plurality of signal measures that are indicative of a current nociception event of the patient and predictive of a future nociception event of the patient. The hemodynamic monitor determines, based on the input features, a nociception score representing a probability of the current nociception event of the patient and/or a probability of the future nociception event of the patient. The hemodynamic monitor invokes a sensory alarm to produce a sensory signal in response to the nociception score satisfying a predetermined level criterion. An analgesic is administered to the patient when the nociception score satisfies the predetermined level criterion.

### BRIEF DESCRIPTION OF THE DRAWINGS

FIG. 1 is a perspective view of an example hemodynamic monitor that analyzes an arterial pressure of a patient and provides a risk score and a warning to medical personnel of a nociception event of the patient.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that determines risk scores representing a probability of a current nociception event, a future nociception event, a current hemodynamic drug administration event, a future hemodynamic drug administration event, and/or a stable period for a patient based on a set of input features derived from signal measures of an arterial pressure waveform of the patient.

FIG. 5 is a diagram of a clinical dataset and with clinical annotations used for data mining and machine training of the hemodynamic monitoring system.

FIG. 6 is a graph illustrating a systolic blood pressure and a heart rate over time from the clinical dataset of FIG. 5 and showing a nociception event and an administration of an analgesic.

FIG. 7 is a graph illustrating a systolic blood pressure and a heart rate over time from the clinical dataset of FIG. 5 and showing a stable episode.

FIG. 8 is a graph illustrating a systolic blood pressure and a heart rate over time from the clinical dataset of FIG. 5 and showing a hemodynamic drug administration event and an administration of a vasopressor drug.

FIG. 9 is a flow diagram for extracting a set of input features derived from waveform features of an arterial pressure waveform of a patient for training a machine learning model of a hemodynamic monitoring system.

FIG. 10 is a graph illustrating an example trace of an arterial pressure waveform including example indicia corresponding to signal measures used to extract the input features that determine the risks scores of the patient.

### DETAILED DESCRIPTION

As described herein, a hemodynamic monitoring system implements a predictive model that produces risk scores representing a probability of a current nociception event for a patient, a probability of a future nociception event for the patient, and a probability that the patient is experiencing a stable episode. The predictive model of the hemodynamic monitoring system can also optionally produce a risk score representing a probability that the patient is experiencing the effects of a previously administered hemodynamic drug (hereinafter referred to as a “hemodynamic drug administration event”) and is not experiencing a current nociception event. The predictive model of the hemodynamic monitoring system can also optionally produce a risk score representing a probability that the patient is experiencing the onset of a future hemodynamic drug administration event and is not experiencing the onset of a future nociception event.

The predictive model of the hemodynamic monitoring system uses machine learning to extract sets of input features from the arterial pressure of the patient. The sets of input features are used by the hemodynamic monitoring system to produce the above-described risk scores for the patient during operation in, e.g., an operating room (OR), an intensive care unit (ICU), or other patient care environment. Depending on the level of the risk scores, the hemodynamic monitoring system can raise a signal or an alarm to medical workers to alert the medical workers that the patient is experiencing a nociception event or will soon be experiencing a nociception event. After receiving the signal, the medical workers can administer an analgesic to the patient to mitigate or prevent the nociception event. If the risk scores determine that the patient is experiencing a hemodynamic drug event, or will soon be experiencing a hemodynamic drug event, the hemodynamic monitoring system can send a signal to the medical workers so that the medical workers do not confuse the hemodynamic drug event of the patient with a nociception event.

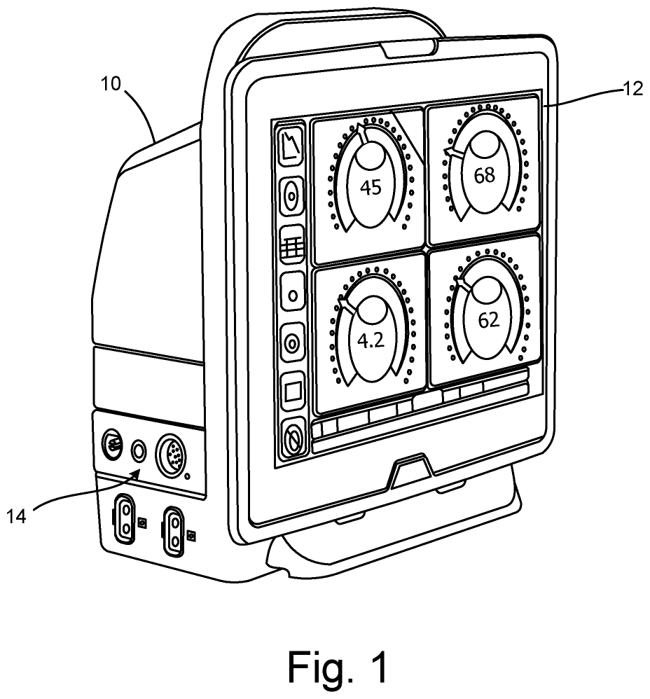

The machine learning of the predictive model of the hemodynamic monitoring system is trained using a clinical data set containing arterial pressure waveforms labeled with clinical annotations of administration of analgesics, vasopressors, inotropes, fluids, and other medication that alter cardiovascular hemodynamics. The hemodynamic monitoring system is described in detail below with reference to FIGS. 1-10.

FIG. 1 is a perspective view of hemodynamic monitor 10 that determines a score representing a probability of a current nociception event of a patient and/or a score representing a probability of a future nociception event for the patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores nociception detection and prediction software code which is executable to produce a score representing a probability of a present (i.e., current) nociception event for a patient and/or a score representing a probability of a future nociception event for the patient. Hemodynamic monitor 10 can receive sensed hemodynamic data representative of an arterial pressure waveform of the patient, such as via one or more hemodynamic sensors connected to hemodynamic monitor 10 via I/O connectors 14. Hemodynamic monitor 10 executes the nociception prediction software code to obtain, using the received hemodynamic data, multiple nociception profiling parameters (e.g., input features), which can include one or more vital sign parameters characterizing vital sign data of the patient, as well as differential and combinatorial parameters derived from the one or more vital sign parameters, as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

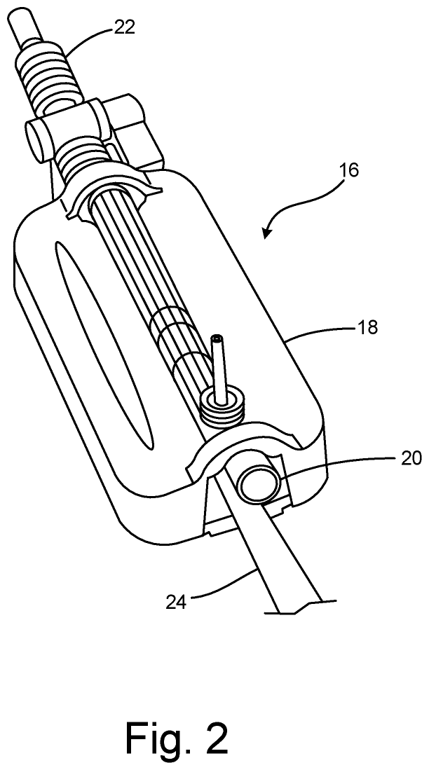

FIG. 2 is a perspective view of hemodynamic sensor 16 that can be attached to a patient for sensing hemodynamic data representative of arterial pressure of the patient. Hemodynamic sensor 16, illustrated in FIG. 2, is one example of a minimally invasive hemodynamic sensor that can be attached to the patient via, e.g., a radial arterial catheter inserted into an arm of the patient. In other examples, hemodynamic sensor 16 can be attached to the patient via a femoral arterial catheter inserted into a leg of the patient.

As illustrated in FIG. 2, hemodynamic sensor 16 includes housing 18, fluid input port 20, catheter-side fluid port 22, and I/O cable 24. Fluid input port 20 is configured to be connected via tubing or other hydraulic connection to a fluid source, such as a saline bag or other fluid input source. Catheter-side fluid port 22 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 24 is configured to connect to hemodynamic monitor 10 via, e.g., one or more of I/O connectors 14 (FIG. 1). Housing 18 of hemodynamic sensor 16 encloses one or more pressure transducers, communication circuitry, processing circuitry, and corresponding electronic components to sense fluid pressure corresponding to arterial pressure of the patient that is transmitted to hemodynamic monitor 10 (FIG. 1) via I/O cable 24.

In operation, a column of fluid (e.g., saline solution) is introduced from a fluid source (e.g., a saline bag) through hemodynamic sensor 16 via fluid input port 20 to catheter-side fluid port 22 toward the catheter inserted into the patient. Arterial pressure is communicated through the fluid column to pressure sensors located within housing 16 which sense the pressure of the fluid column. Hemodynamic sensor 16 translates the sensed pressure of the fluid column to an electrical signal via the pressure transducers and outputs the corresponding electrical signal to hemodynamic monitor 10 (FIG. 1) via I/O cable 24. Hemodynamic sensor 16 therefore transmits analog sensor data (or a digital representation of the analog sensor data) to hemodynamic monitor 10 (FIG. 1) that is representative of substantially continuous beat-to-beat monitoring of the arterial pressure of the patient.

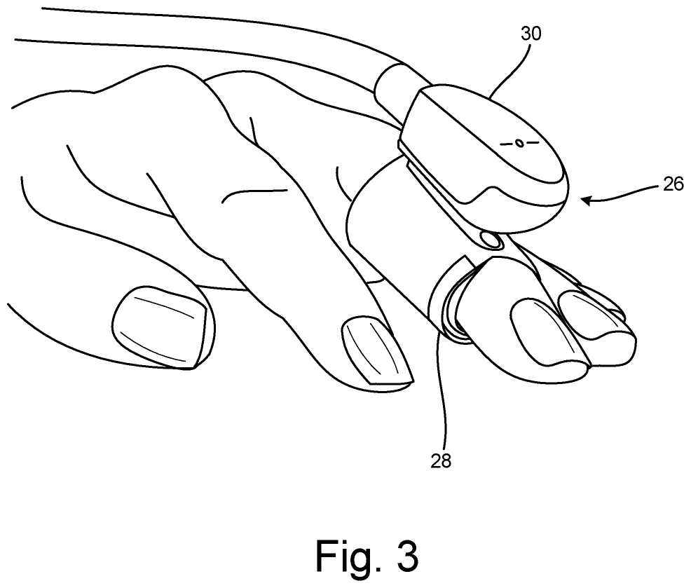

FIG. 3 is a perspective view of hemodynamic sensor 26 for sensing hemodynamic data representative of arterial pressure of a patient. Hemodynamic sensor 26, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 26 includes inflatable finger cuff 28 and heart reference sensor 30. Inflatable finger cuff 28 includes an inflatable blood pressure bladder configured to inflate and deflate as controlled by a pressure controller (not illustrated) that is pneumatically connected to inflatable finger cuff 28. Inflatable finger cuff 28 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 28. The pressure applied by the pressure controller to continuously maintain the unloaded volume is representative of the blood pressure in the finger, and is communicated by the pressure controller to hemodynamic monitor 10 shown in FIG. 1. Heart reference sensor 30 measures the hydrostatic height difference between the level at which the finger is kept and the reference level for the pressure measurement, which typically is heart level. Accordingly, hemodynamic sensor 26 transmits sensor data that is representative of substantially continuous beat-to-beat monitoring of the arterial pressure waveform of the patient.

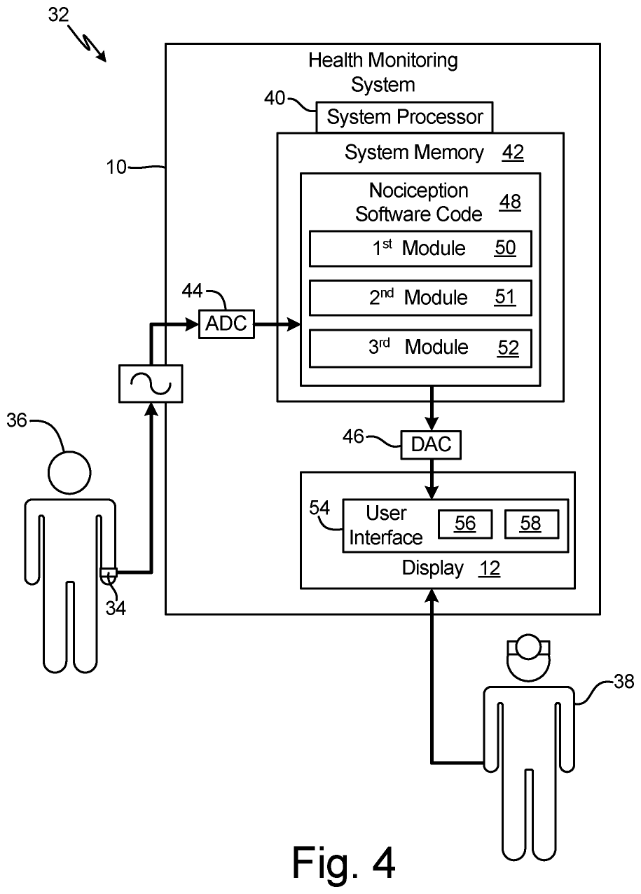

FIG. 4 is a block diagram of hemodynamic monitoring system 32 that determines a nociception score representing a probability of a current or future nociception event of patient 36 based on a set of nociception profiling parameters (also referred to as input features) derived from the arterial pressure of the patient. Hemodynamic monitoring system 32 monitors the arterial pressure of patient 36 and provides a warning to medical worker 38 when the nociception score of patient 36 rises above a predetermined threshold. Medical worker 38 can respond to the warning by administering an appropriate analgesic to patient 36 to mitigate the current or future nociception event.

As illustrated in FIG. 4, hemodynamic monitoring system 32 includes hemodynamic monitor 10 and hemodynamic sensor 34. Hemodynamic monitoring system 32 can be implemented within a patient care environment, such as an ICU, an OR, or other patient care environment. As illustrated in FIG. 4, the patient care environment can include patient 36 and healthcare worker 38 trained to utilize hemodynamic monitoring system 32.

Hemodynamic monitor 10, as described above with respect to FIG. 1, can be, e.g., an integrated hardware unit including system processor 40, system memory 42, display 12, analog-to-digital (ADC) converter 44, and digital-to-analog (DAC) converter 46. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. In general, though illustrated and described in the example of FIG. 4 as an integrated hardware unit, it should be understood that hemodynamic monitor 10 can include any combination of devices and components that are electrically, communicatively, or otherwise operatively connected to perform functionality attributed herein to hemodynamic monitor 10.

As illustrated in FIG. 4, system memory 42 stores nociception software code 48 which forms the predictive model of hemodynamic monitor 10. Nociception software code 48 includes first module 50 for extracting and calculating waveform features from the arterial pressure of patient 36, second module 51 for extracting input features from the waveform features, and third module 52 for calculating probability of nociception of patient 36 based on the input features. Display 12 provides user interface 54, which includes control elements 56 that enable user interaction with hemodynamic monitor 10 and/or other components of hemodynamic monitoring system 32. User interface 54, as illustrated in FIG. 4, also provides sensory alarm 58 to provide warning to medical personnel of a current nociception event or a predicted future nociception event of patient 36, as is further described below. Sensory alarm 58 can be implemented as one or more of a visual alarm, an audible alarm, a haptic alarm, or other type of sensory alarm. For instance, sensory alarm 58 can be invoked as any combination of flashing and/or colored graphics shown by use interface 54 on display 12, display of the nociception score via user interface 54 on display 12, a warning sound such as a siren or repeated tone, and a haptic alarm configured to cause hemodynamic monitor 10 to vibrate or otherwise deliver a physical impulse perceptible to medical worker 38 or other user.

Hemodynamic sensor 34 can be attached to patient 36 to sense hemodynamic data representative of the arterial pressure waveform of patient 36. Hemodynamic sensor 34 is operatively connected to hemodynamic monitor 10 (e.g., electrically and/or communicatively connected via wired or wireless connection, or both) to provide the sensed hemodynamic data to hemodynamic monitor 10. In some examples, hemodynamic sensor 34 provides the hemodynamic data representative of the arterial pressure waveform of patient 36 to hemodynamic monitor 10 as an analog signal, which is converted by ADC 44 to digital hemodynamic data representative of the arterial pressure waveform. In other examples, hemodynamic sensor 34 can provide the sensed hemodynamic data to hemodynamic monitor 10 in digital form, in which case hemodynamic monitor 10 may not include or utilize ADC 44. In yet other examples, hemodynamic sensor 34 can provide the hemodynamic data representative of the arterial pressure waveform of patient 36 to hemodynamic monitor 10 as an analog signal, which is analyzed in its analog form by hemodynamic monitor 10.

Hemodynamic sensor 34 can be a non-invasive or minimally invasive sensor attached to patient 36. For instance, hemodynamic sensor 34 can take the form of minimally invasive hemodynamic sensor 16 (FIG. 2), non-invasive hemodynamic sensor 26 (FIG. 3), or other minimally invasive or non-invasive hemodynamic sensor. In some examples, hemodynamic sensor 34 can be attached non-invasively at an extremity of patient 36, such as a wrist, an arm, a finger, an ankle, a toe, or other extremity of patient 36. As such, hemodynamic sensor 34 can take the form of a small, lightweight, and comfortable hemodynamic sensor suitable for extended wear by patient 36 to provide substantially continuous beat-to-beat monitoring of the arterial pressure of patient 36 over an extended period of time, such as minutes or hours.

In certain examples, hemodynamic sensor 34 can be configured to sense an arterial pressure of patient 36 in a minimally invasive manner. For instance, hemodynamic sensor 34 can be attached to patient 36 via a radial arterial catheter inserted into an arm of patient 36. In other examples, hemodynamic sensor 34 can be attached to patient 36 via a femoral arterial catheter inserted into a leg of patient 36. Such minimally invasive techniques can similarly enable hemodynamic sensor 34 to provide substantially continuous beat-to-beat monitoring of the arterial pressure of patient 36 over an extended period of time, such as minutes or hours.

System processor 40 is a hardware processor configured to execute nociception software code 48, which implements first module 50, second module 51, and third module 52 to produce a nociception score representing a probability of a current nociception event or a probability of a future nociception event for patient 36. Examples of system processor 40 can include any one or more of a microprocessor, a controller, a digital signal processor (DSP), an application specific integrated circuit (ASIC), a field-programmable gate array (FPGA), or other equivalent discrete or integrated logic circuitry.

System memory 42 can be configured to store information within hemodynamic monitor 10 during operation. System memory 42, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 42 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 54 can include graphical and/or physical control elements that enable user input to interact with hemodynamic monitor 10 and/or other components of hemodynamic monitoring system 32. In some examples, user interface 54 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 54 can take the form of and/or include physical control elements, such as a physical buttons, keys, knobs, or other physical control elements configured to receive user input to interact with components of hemodynamic monitoring system 32.

In operation, hemodynamic sensor 34 senses hemodynamic data representative of an arterial pressure waveform of patient 36. Hemodynamic sensor 34 provides the hemodynamic data (e.g., as analog sensor data), to hemodynamic monitor 10. ADC 44 converts the analog hemodynamic data to digital hemodynamic data representative of the arterial pressure waveform of the patient.

Nociception software code 48 can include nociception detection software code. System processor 40 executes the nociception detection software code of nociception software code 48 to determine, using the received hemodynamic data, a nociception detection score representing a probability of a current nociception event for patient 36. For instance, system processor 40 can execute first module 50 to perform waveform analysis of the hemodynamic data to determine a plurality of signal measures. System processor 40 executes second module 51 to extract nociception detection input features from the plurality of signal measures that detect the nociception event of patient 36. System processor 40 executes third module 52 to determine, based on the nociception detection input features, a nociception detection score representing a probability of the nociception event of patient 36. If the nociception detection score satisfies a predetermined detection criterion, system processor 40 invokes sensory alarm 58 of user interface 54 to send a first sensory signal to alert medical worker 38 that patient 36 is presently experiencing a current nociception event. Medical worker 38 can respond to the warning by administering an analgesic to patient 36, or administering any other form of treatment to patient 36, to mitigate the current nociception event.

Nociception software code 48 can also include nociception prediction software code. System processor 40 executes the nociception prediction software code of nociception software code 48 to determine, using the received hemodynamic data, a nociception prediction score representing a probability of a future nociception event for patient 36. For instance, system processor 40 can execute first module 50 to perform waveform analysis of the hemodynamic data to determine the plurality of signal measures. System processor 40 executes second module 51 to extract nociception prediction input features from the plurality of signal measures that predict the future nociception event of patient 36. System processor 40 executes third module 52 to determine, based on the nociception prediction input features, a nociception prediction score representing a probability of the future nociception event of patient 36. If the nociception prediction score satisfies a predetermined prediction criterion, system processor 40 invokes sensory alarm 58 of user interface 54 to send a second sensory signal to alert medical worker 38 that patient 36 will soon be experiencing a future nociception event. Medical worker 38 can respond to this warning by administering an analgesic to patient 36, or administering any other form of treatment to patient 36, to mitigate or prevent the onset of the predicted future nociception event.

In addition to detecting current nociception events and predicting future nociception events of patient 36, hemodynamic monitoring system 32 can discern when patient 36 is experiencing a current nociception event and when patient 36 is merely reacting to a hemodynamic drug previously administered to patient 36 by medical worker 38 (hereinafter referred to as a hemodynamic drug administration event). The hemodynamic drug administration event is defined as an event where patient 36 experiences an increase in heart rate and an increase in blood pressure due to the administration of a compound that alters cardiovascular hemodynamics (e.g., analgesics, vasopressors, inotropes, fluids, and/or other medication) and is not a nociception event of patient 36. Nociception software code 48 includes hemodynamic drug detection software code for detecting the presence of hemodynamic drug administration event of patient 36. System processor 40 executes the hemodynamic drug detection software code of nociception software code 48 to determine, using the received hemodynamic data, a hemodynamic drug detection score representing a probability that the hemodynamic drug administration event is responsible for increasing a heart rate and a blood pressure of patient 36. For instance, system processor 40 can execute first module 50 to perform waveform analysis of the hemodynamic data to determine the plurality of signal measures. System processor 40 executes second module 51 to extract hemodynamic drug detection input features from the plurality of signal measures that detect the current effects of the hemodynamic drug administration event of patient 36. System processor 40 executes third module 52 to determine, based on the hemodynamic drug detection input features, the hemodynamic drug detection score of patient 36. If the hemodynamic drug detection score satisfies a predetermined hemodynamic detection criterion, system processor 40 invokes sensory alarm 58 of user interface 54 to send a third sensory signal to alert medical worker 38 that patient 36 is experiencing a hemodynamic drug administration event and not a current nociception event. The hemodynamic drug detection score and the third sensory signal help to prevent medical worker 38 from confusing the hemodynamic drug administration event with a nociception event and prevent medical worker 38 from unnecessarily administering analgesics to patient 36.

Nociception software code 48 also includes hemodynamic drug prediction software code for detecting the onset of a future hemodynamic drug administration event of patient 36. System processor 40 executes the hemodynamic drug prediction software code of nociception software code 48 to determine, using the received hemodynamic data, a hemodynamic drug prediction score representing a probability that the hemodynamic drug administration event is responsible for increasing a heart rate and a blood pressure of patient 36. For instance, system processor 40 can execute first module 50 to perform waveform analysis of the hemodynamic data to determine the plurality of signal measures. System processor 40 executes second module 51 to extract hemodynamic drug prediction input features from the plurality of signal measures that detect the onset of the hemodynamic drug administration event of patient 36. System processor 40 executes third module 52 to determine, based on the hemodynamic drug prediction input features, the hemodynamic drug prediction score of patient 36. If the hemodynamic drug prediction score satisfies a predetermined hemodynamic prediction criterion, system processor 40 invokes sensory alarm 58 of user interface 54 to send a fourth sensory signal to alert medical worker 38 that patient 36 will soon be experiencing a hemodynamic drug administration event. The hemodynamic drug prediction score and the fourth sensory signal help to prevent medical worker 38 from confusing the future hemodynamic drug administration event with a future nociception event and prevent medical worker 38 from unnecessarily administering analgesics to patient 36.

System memory 42 of hemodynamic monitor 10 can also include stable detection software code for detecting a stable episode of patient 36. The stable episode is defined as a period during which patient 36 does not experience a nociception event or a hemodynamic drug administration event. The stable detection software code can be a subpart of nociception software code 48. System processor 40 executes stable detection software code to extract stable detection input features from the plurality of signal measures. Stable detection software code can extract the stable detection input features from the plurality of signal measures using second module 51. The stable detection input features detect the stable episode of patient 36. System processor 40 executes third module 52 to determine, based on the stable detection input features, a stable score of patient 36. System processor 40 outputs the stable score of patient 36 to user interface 54 of display 12.

System processor 40 can execute first module 50 to extract a single batch of the plurality of signal measures for a given unit of time, and that single batch of signal measures can be used by second module 51 to extract all of nociception detection input features, nociception prediction input features, hemodynamic drug detection input features, hemodynamic drug prediction input features, and stable detection input features for that unit of time. Second module 51 can extract all of nociception detection input features, nociception prediction input features, hemodynamic drug detection input features, hemodynamic drug prediction input features, and stable detection input features concurrently from the plurality of signal measures. System processor 40 can execute third module 52 to concurrently determine the nociception detection score, the nociception prediction score, the hemodynamic drug detection score, the hemodynamic drug prediction score, and the stable score. Nociception software code 48 of hemodynamic monitor 10 can utilize, in some examples, a classification-type machine learning model with binary positive versus negative labels. Processor 40 can, in certain examples, output the nociception detection score and the hemodynamic drug detection score together to display 12 to compare and contrast the two probabilities and help medical worker 38 better understand whether a nociception event or a hemodynamic drug administration event is causing the increase in blood pressure and heart rate in patient 36.

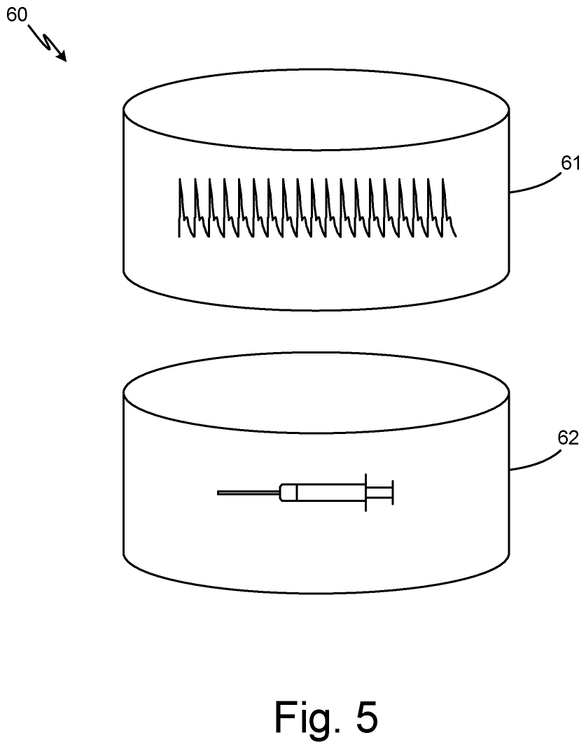

Alternatively, nociception software code 48 of hemodynamic monitor 10 can utilize a multi-class machine learning model with three labels: nociception event verses hemodynamic drug event verses stable episode. For example, processor 40 can output to display 12 the nociception detection score with both the stable score and the hemodynamic drug detection score, so that all three probabilities are compared together: the probability the patient is undergoing a current nociception event, the probability that the patient is experiencing a current hemodynamic drug administration event, and the probability that the patient is stable. As discussed below with reference to FIGS. 5-8, the machine learning model of hemodynamic monitor 10 can be trained to recognize and/or predict these types of events and episodes in the arterial pressure waveform of patient 36 using a clinical dataset containing arterial pressure waveforms and clinical annotations of administration of a compound that alters cardiovascular hemodynamics (e.g., analgesics, vasopressors, inotropes, fluids, and/or other medication).

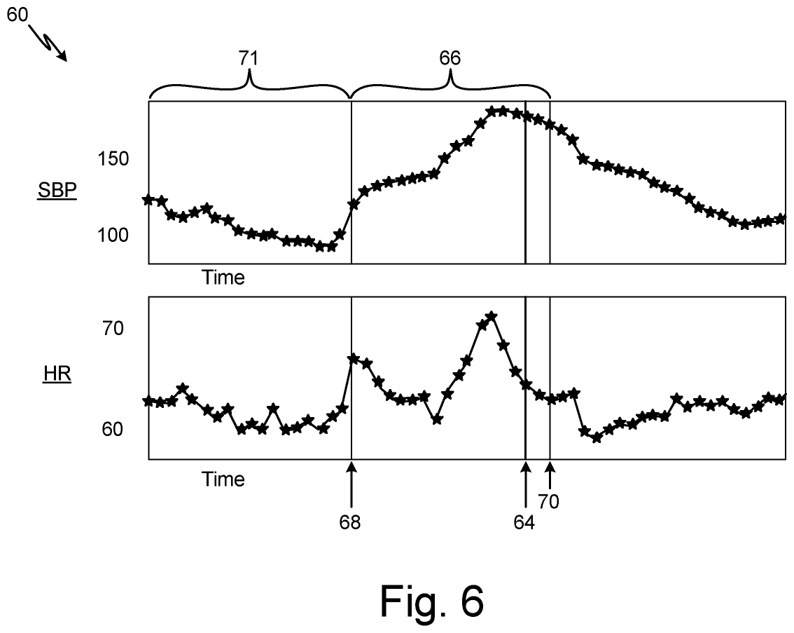

FIG. 5 is a diagram of clinical dataset 60 used for data mining and machine training of the hemodynamic monitor 10. Clinical dataset 60 includes first data set 61 containing a collection of arterial pressure waveforms recorded from previous patients. First data set 61 can be collected by invasive hemodynamic sensors, such as hemodynamic sensor 16 shown in FIG. 2, or collected by non-invasive hemodynamic sensors, such as hemodynamic sensor 26 shown in FIG. 3. Clinical dataset 60 also includes second data set 62 containing a log of instances where compounds that alter cardiovascular hemodynamics (e.g., analgesics, vasopressors, inotropes, fluids, and/or other medication) were administered to the previous patients of first data set 61 while their arterial pressure waveforms were being recorded. Medical workers can enter the administration information directly into the same hemodynamic monitors that are collecting first data set 61, such that first data set 61 and second data set 62 are collected together concurrently. As shown in FIGS. 6-8, the information in second data set 62 is annotated and labeled onto the collection of arterial pressure waveforms of first data set 61.

FIG. 6 is a graph illustrating a plot of systolic blood pressure over time (hereinafter referred to as “SBP plot”) and a plot of heart rate over time (hereinafter referred to as “HR plot”). Before clinical dataset 60 can be used to machine train hemodynamic monitor 10, the SBP plot and the HR plot are determined for each of the arterial pressure waveforms collected in clinical dataset 60. The SBP plot and the HR plot shown in FIG. 6 are an example from one of the arterial pressure waveforms (not shown) in clinical dataset 60. After the SBP plot and the HR plot are determined for each of the arterial pressure wave forms collected in clinical dataset 60, the SBP plot and the HR plot are both annotated to show when compounds that alter the cardiovascular hemodynamics were administered to the clinical patient. For example, the SBP plot and the HR plot shown in FIG. 6 include analgesic label 64, which is a vertical bar extending across the SBP plot and the HR plot at the same position in time. Analgesic label 64 in FIG. 6 indicates that the clinical patient was administered an analgesic during the time represented by the SBP plot and the HR plot in FIG. 6. After the SBP plot and the HR plot are annotated and labeled to show drug administrations to the clinical patient, nociception data segments 66 are identified and labeled on the SBP plot and the HR plot.

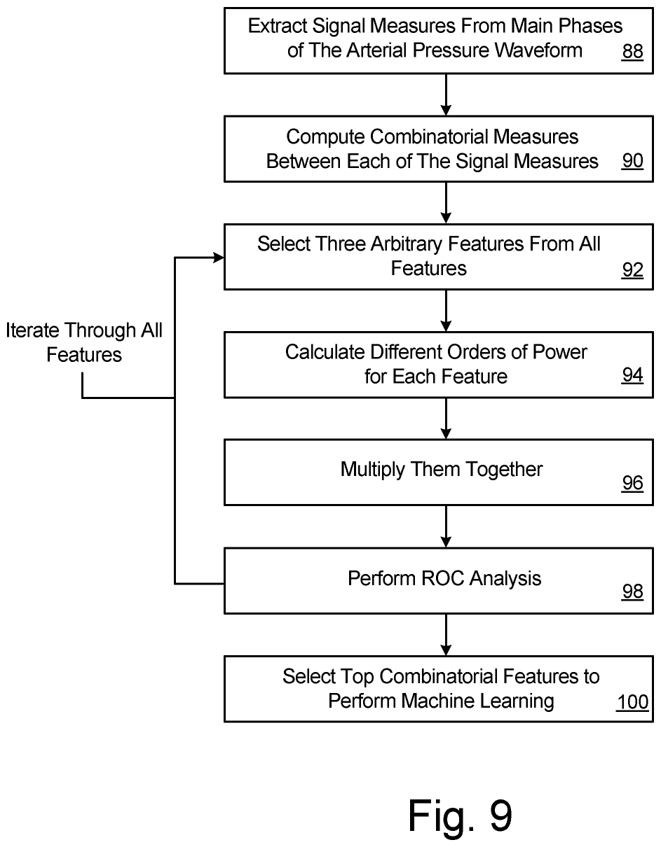

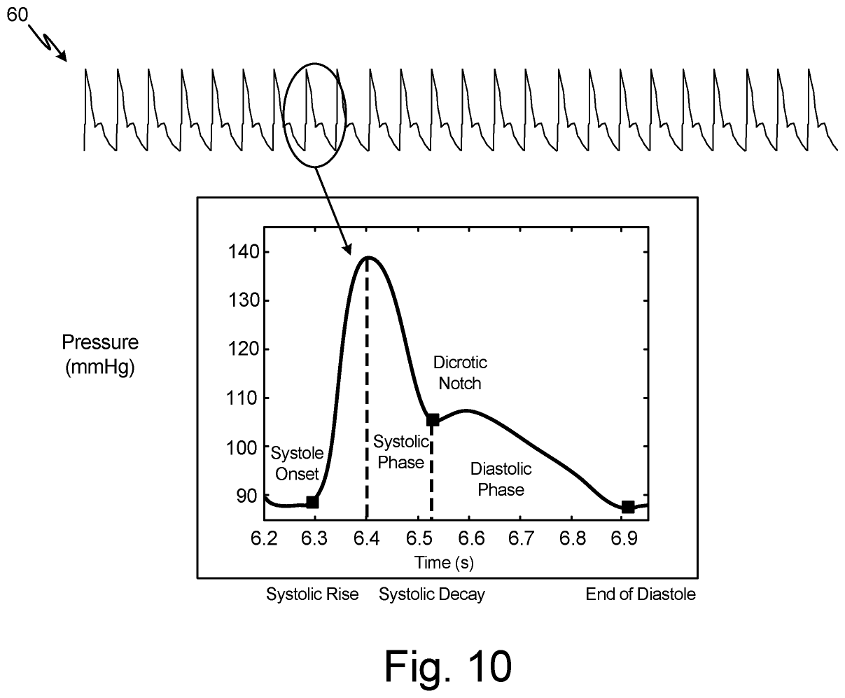

As shown in FIG. 6, nociception data segments 66 are identified on the SBP plot and the HR plot by locating time segments in both the SBP plot and the HR plot where systolic blood pressure of the clinical patient increases by at least a threshold amount (e.g., 20% or other threshold amounts) compared to a prior time period, where heart rate of the clinical patient also increases by at least a threshold amount (e.g. 20% or other threshold amounts) compared to the prior time period, and there has been no infusion of a compound that alters cardiovascular hemodynamics (e.g., analgesics, vasopressors, inotropes, fluids, and/or other medication) started prior to the increase in blood pressure and the increase in heart rate. In FIG. 6, both the SBP plot and the HR plot increase by more than 20% at starting point 68, which occurs before analgesic label 64, thus indicating the start of nociception data segment 66 in FIG. 6. Nociception data segment 66 in FIG. 6 continues until both the SBP plot and the HR plot begin to drop as a result of the analgesic administered in time to the clinical patient at analgesic label 64. The drops in the SBP plot and the HR plot are indicated by ending point 70. Starting point 68 and ending point 70 are both labeled on the HR plot and the SBP plot and the time segment between starting point 68 and ending point 70 is designated as one nociception data segment 66. With analgesic label 64 and nociception data segment 66 identified on the SBP plot and the HR plot, the arterial pressure waveform used to generate the SBP plot and the HR plot in FIG. 6 can also be annotated and labeled to show when analgesic label 64 and nociception data segment 66 occurred on the arterial pressure waveform. Once labeled with analgesic label 64 and nociception data segment 66, the arterial pressure waveform is ready to be used for data mining and machine training of the hemodynamic monitor 10 to detect nociception events. As will be discussed further below with reference to FIGS. 9-10, waveform analysis is performed on clinical data set 60 containing nociception data segments 66 to calculate a plurality of signal measures which are then used to compute the nociception detection input features that best detect the probability of current nociception events.

Prediction data segments 71 in FIG. 6 can also be used for machine training hemodynamic monitor 10 to predict future nociception events. Prediction data segments 71 can be identified in clinical dataset 60 by identifying the prior time period before the start of the increase in the SBP plot and the HR plot. In the example of FIG. 6, the prior time period occurs before starting point 68. The prior time period before starting point 68 is labeled as a prediction data segment 71. In some examples, prediction data segment 71 includes a time period starting fifteen-minutes before nociception data segment 66 and ending immediately prior to nociception data segment 66. In other examples, prediction data segment 71 can include a larger or smaller time period before nociception data segment 66. With prediction data segment 71 identified and labeled on the SBP plot and the HR plot, the arterial pressure waveform used to generate the SBP plot and the HR plot in FIG. 6 can also be annotated and labeled to show when prediction data segment 71 occurred on the arterial pressure waveform. Once labeled with prediction data segment 71, the arterial pressure waveform is ready to be used for data mining and machine training of the hemodynamic monitor 10 to predict nociception events. As will be discussed further below with reference to FIGS. 9-10, waveform analysis is performed on clinical data set 60 containing prediction data segments 71 to calculate a plurality of signal measures which are then used to compute the nociception prediction input features that best detect the probability of the onset of a future nociception event.

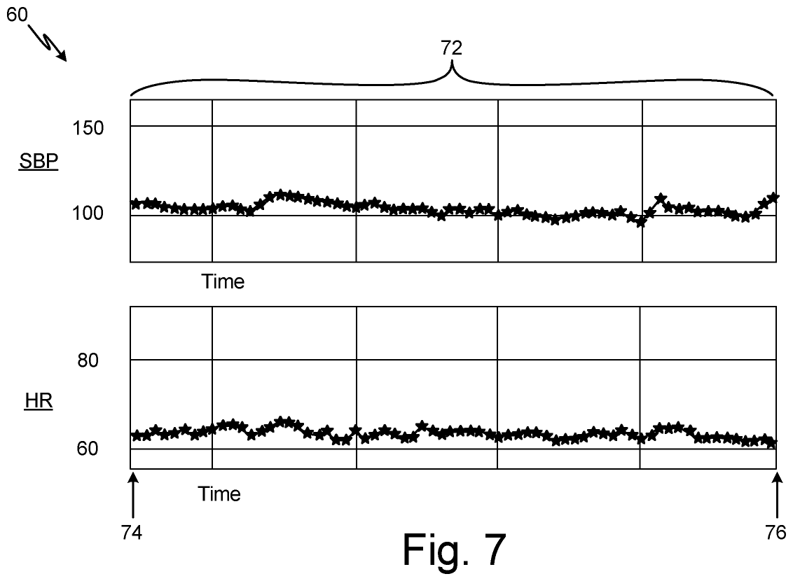

FIG. 7 is a graph illustrating another SBP plot and HR plot derived from an arterial pressure waveform segment (not shown) from clinical data set 60. The arterial pressure waveform segment that produced the SBP plot and the HR plot in FIG. 7 can be identified as a stable data segment 72 and used for data mining and machine training hemodynamic monitor 10 to detect when a patient is experiencing a stable episode with no nociception. An arterial pressure waveform segment in clinical data set 60 is identified as stable data segment 72 if there is no increase greater than a threshold mount (e.g. 20% or other threshold amounts) in the SBP plot, no increase greater than a threshold amount (e.g. 20% or other threshold amount) in the HR plot, and no infusion performed of a compound that alters cardiovascular hemodynamics. As shown in the example of FIG. 7, the SBP plot does not include an increase greater than 20% between starting point 74 and ending point 76. The HR plot in the example of FIG. 7 also does not include an increase greater than 20% between starting point 74 and ending point 76. The HR plot and the SBP plot of FIG. 7 also does not include any annotations or labels indicating an infusion of a compound that alters cardiovascular hemodynamics in the clinical patient between starting point 74 and ending point 76. Given the above-described characteristics of the HR plot and the SBP plot in the example of FIG. 7, the HR plot and the SBP plot of FIG. 7 are labeled as a stable data segment 72 between starting point 74 and ending point 76. The arterial pressure waveform segment (not shown) that generated the HR plot and the SBP plot of FIG. 7 is also labeled as a stable data segment 72 between starting point 74 and ending point 76. Once labeled with stable data segment 72, the arterial pressure waveform segment is ready to be used for stable data mining and stable machine training of the hemodynamic monitor 10. As will be discussed further below with reference to FIGS. 9-10, waveform analysis is performed on clinical data set 60 containing stable data segments 72 to calculate a plurality of signal measures which are then used to compute the stable detection input features that best detect the probability of stable episodes.

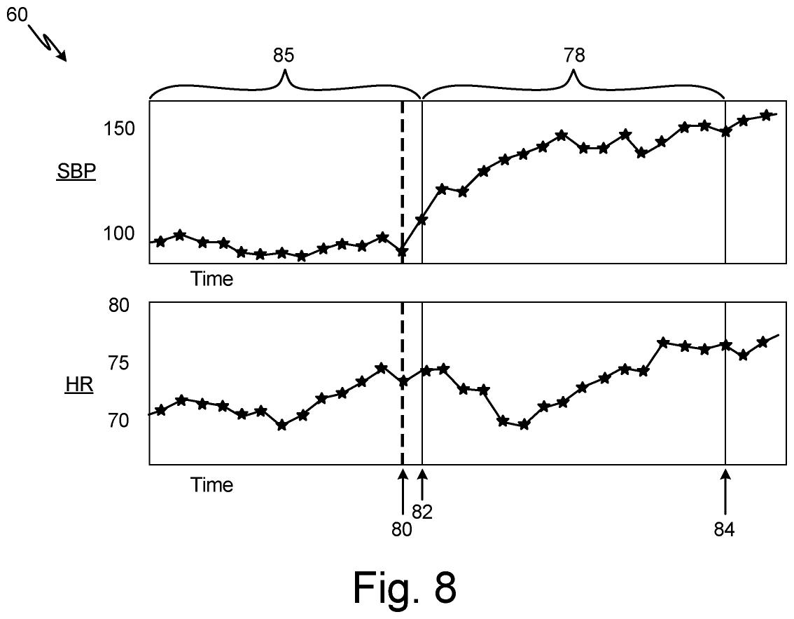

FIG. 8 is a graph illustrating another SBP plot and HR plot derived from an arterial pressure waveform segment (not shown) from clinical data set 60. The arterial pressure waveform segment that produced the SBP plot and HR plot in FIG. 8 can be identified as a hemodynamic drug administration data segment 78 (referred to hereinafter as “HDA data segment 78”) and used for data mining and machine training hemodynamic monitor 10 to detect when a patient is experience a current hemodynamic drug administration event. An arterial pressure waveform segment in clinical data set 60 is identified as a HDA data segment 78 if the arterial pressure waveform segment includes an infusion of a compound that alters cardiovascular hemodynamics into the clinical patient and there is an increase of at least at least a threshold amount (e.g. 20% or other threshold amounts) in both the SBP plot and the HR plot after the infusion. In the example of FIG. 8, vasopressor infusion label 80 on the SBP plot and the HR plot indicates that the clinical patient was administered a vasopressor drug. Shortly after vasopressor infusion label 80, the SBP plot increased by at least 20% between starting point 82 and ending point 84. Between starting point 82 and ending point 84, HR plot also increased by at least 20%, thus indicating that a HDA data segment 78 occurred between starting point 82 and ending point 84. The HR plot and the SBP plot of FIG. 8 are labeled as a HDA data segment 78 between starting point 82 and ending point 84. The arterial pressure waveform segment (not shown) that generated the HR plot and the SBP plot of FIG. 8 is also labeled as a HDA data segment 78 between starting point 82 and ending point 84. Once labeled with HDA data segment 78, the arterial pressure waveform segment is ready to be used for hemodynamic drug detection data mining and machine training of the hemodynamic monitor 10. As will be discussed further below with reference to FIGS. 9-10, waveform analysis is performed on clinical data set 60 containing HDA data segments 78 to calculate a plurality of signal measures which are then used to compute the hemodynamic drug detection input features that best detect the probability of current hemodynamic drug administration events.

Hemodynamic drug prediction data segments 85 in FIG. 8 (referred hereinafter as “HDP data segments 85”) can also be used for machine training hemodynamic monitor 10 to predict future hemodynamic drug administration events. HDP data segments 85 can be identified in clinical dataset 60 by identifying a previous time period before the start of the increase in the SBP plot and the HR plot in FIG. 8. In the example of FIG. 8, the previous time period occurs before starting point 82. The previous time period before starting point 82 is labeled as a HDP data segment 85. In some examples, HDP data segment 85 includes a time period starting fifteen-minutes before HDA data segment 78 and ending immediately prior to HDA data segment 78. In other examples, HDP data segment 85 can include a larger or smaller time period before HDA data segment 78. With HDP data segment 85 identified and labeled on the SBP plot and the HR plot, the arterial pressure waveform used to generate the SBP plot and the HR plot in FIG. 8 can also be annotated and labeled to show when HDP data segment 85 occurred on the arterial pressure waveform. Once labeled with HDP data segment 85, the arterial pressure waveform is ready to be used for data mining and machine training of the hemodynamic monitor 10 to predict the onset of future hemodynamic drug administration events. As will be discussed further below with reference to FIGS. 9-10, waveform analysis is performed on clinical data set 60 containing HDP data segments 85 to calculate a plurality of signal measures which are then used to compute the hemodynamic drug prediction input features that best detect the probability of the onset of a future hemodynamic drug administration event.

FIG. 9 is a flow diagram of method 86 for data mining clinical data set 60 from FIGS. 5-8 for machine training the machine learning model of hemodynamic monitor 10. Method 86 in FIG. 9 will be discussed while also referencing FIG. 10. Method 86 is applied to each of nociception data segments 66, prediction data segments 71, stable data segments 72, HDA data segments 78, and HDP data segments 85 in clinical data set 60 to train hemodynamic monitor to find the input features previously described with reference to FIG. 4. Method 86 will be described as applied to nociception data segments 66 (shown in FIG. 6).

To machine train hemodynamic monitor 10 to identify the nociception detection input features described in FIG. 4, nociception detection input features are first determined by applying method 86 to nociception data segments 66 of clinical data set 60. First step 88 of method 86 is to perform waveform analysis of nociception data segments 66 of the arterial waveforms collected in data set 60 to calculate a plurality of signal measures of the nociception data segments. Performing waveform analysis of nociception data segments 66 can include identifying individual cardiac cycles in each of the arterial pressure waveforms of nociception data segments 66. FIG. 10 provides an example graph illustrating an example trace of an arterial pressure waveform with an individual cardiac cycle identified and enlarged. Next, performing waveform analysis of nociception data segments 66 can include identifying a dicrotic notch in each of the individual cardiac cycles of each of the arterial pressure waveforms of nociception data segments 66, similar to the example shown in FIG. 10. Next, the waveform analysis on nociception data segments 66 includes identifying a systolic rise phase, a systolic decay phase, and a diastolic phase in each of the individual cardiac cycles of each of the arterial pressure waveforms of nociception data segments 66, similar to the example shown in FIG. 10.

Signal measures are extracted from each of the systolic rise phase, the systolic decay phase, and the diastolic phase from each of the individual cardiac cycles of each of the arterial pressure waveforms of nociception data segments 66. The signal measures can correspond to hemodynamic effects from each of the systolic rise phase, the systolic decay phase, and the diastolic phase from each of the individual cardiac cycles. Those hemodynamic effects can include contractility, aortic compliance, stroke volume, vascular tone, afterload, and full cardiac cycle. The signal measures calculated or extracted by the waveform analysis of first step 88 of method 86 includes a mean, a maximum, a minimum, a duration, an area, a standard deviation, derivatives, and/or morphological measures from each of the systolic rise phase, the systolic decay phase, and the diastolic phase from each of the individual cardiac cycles. The signal measures can also include heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles of each of the arterial pressure waveforms of nociception data segments 66.

After the signal measures are determined for nociception data segments 66, step 90 of method 86 is performed on the signal measures of nociception data segments 66. Step 90 of method 86 computes combinatorial measures between the signal measures of nociception data segments 66. Computing the combinatorial measures between the signal measures of nociception data segments 66 can include performing steps 92, 94, 96, and 98 shown in FIG. 9 on all the signal measures of nociception data segments 66. Step 92 is performed by arbitrarily selecting three signal measures from the signal measures of the nociception data segments. Next, different orders of power are calculated for each of the three signal measures to generate powers of the three signal measures, as shown in step 94 of FIG. 9. In step 96 of FIG. 9, the powers of the three signal measures are then multiplied together to generate the product of the powers of the three signal measures. Step 98 includes performing receiver operating characteristic (ROC) analysis of the product to arrive at a combinatorial measure for the three signal measures. Steps 92, 94, 96, and 98 are repeated until all of the combinatorial measures have been computed between all of the signal measures of nociception data segments 66. The signal measures with most predictive top combinatorial measures (i.e., combinatorial measures satisfying a threshold prediction criteria) are selected as top signal measures for nociception data segments 66 and are labeled as the nociception detection input features. With the nociception detection input features determined, hemodynamic monitor 10 is trained or programmed to perform waveform analysis on the arterial pressure waveform of patient 36 (shown in FIG. 4) and extract the nociception detection input features from the arterial pressure waveform of patient 36, and use those nociception detection input features to determine the probability that patient 36 is currently experiencing current nociception event.

Similar to how method 86 was applied to nociception data segments 66, method 86 is applied to prediction data segments 71, stable data segments 72, HDA data segments 78, and HDP data segments 85 in clinical data set 60 to determine the nociception prediction input features, the stable detection input features, the hemodynamic drug detection input features, and the hemodynamic drug prediction input features respectively.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A hemodynamic monitor for detecting nociception of a patient, the hemodynamic monitor comprising:
a non-invasive blood pressure sensor comprising an inflatable blood pressure bladder, a pressure controller pneumatically connected to the inflatable blood pressure bladder, and an optical transmitter and an optical receiver that are electrically connected to the pressure controller; and
an integrated hardware unit comprising:
a system processor;
a system memory; and
a display comprising a user interface; and
wherein the system memory comprises nociception detection instructions that, when executed by the system processor, are configured to:
adjust, by the pressure controller, a pressure within the inflatable blood pressure bladder to maintain a constant volume of an artery of a patient for a period of time based on a feedback signal generated by the optical transmitter and the optical receiver;
generate an arterial pressure waveform data of the patient based on the adjusted pressure within the inflatable blood pressure bladder over the period of time;
extract a plurality of signal measures from the arterial pressure waveform data of the patient;
extract detection input features from the plurality of signal measures that are indicative of a current nociception event of the patient;
determine a nociception score of the patient based on the detection input features;
generate a first sensory alarm signal configured to generate a first sensory alert that indicates that the patient is experiencing the current nociception event when the nociception score satisfies a predetermined detection criterion;
transmit the first sensory alarm signal to the user interface; and
output the first sensory alert through the user interface.

2. The hemodynamic monitor of claim 1, wherein the detection input features of the nociception detection instructions are determined by detection machine training, wherein the detection machine training comprises:
collecting a clinical dataset containing arterial pressure waveforms and clinical annotations of administrations of a compound that alters cardiovascular hemodynamics;
identifying nociception data segments in the clinical dataset, wherein the nociception data segments each comprise:
an increase in blood pressure of at least a first threshold compared to a prior time period;
an increase in heart rate of at least a second threshold compared to the prior time period; and
no infusion of the compound that alters cardiovascular hemodynamics started prior to the increase in blood pressure and the increase in heart rate;
identifying a start and an end of the increase in the blood pressure and the increase in the heart rate;
labeling the nociception data segments after the start and during the increase in the blood pressure and the increase in the heart rate;
performing waveform analysis of the labeled nociception data segments to calculate a plurality of signal measures of the nociception data segments; and
determining the detection input features by computing combinatorial measures between the plurality of signal measures of the nociception data segments and selecting top signal measures from the plurality of signal measures of the nociception data segments with most predictive combinatorial measures and labeling the top signal measures as the detection input features.

3. The hemodynamic monitor of claim 2, wherein the system memory comprises nociception prediction instructions that, when executed by the system processor, are configured to:
extract prediction input features from the plurality of signal measures that are predictive of a future nociception event of the patient, wherein the prediction input features and the detection input features are extracted concurrently from the plurality of signal measures;
determine a nociception prediction score of the patient based on the prediction input features, wherein the nociception prediction score and the nociception score are determined concurrently;
generate a second sensory alarm signal configured to generate a second sensory alert that indicates that the patient is likely to experience the future nociception event when the nociception prediction score satisfies a predetermined prediction criterion;
transmit the second sensory alarm signal to the user interface; and
output the second sensory alert through the user interface.

4. The hemodynamic monitor of claim 3, wherein the prediction input features of the nociception prediction instructions are determined by prediction machine training, wherein the prediction machine training comprises:
identifying the prior time period before the start of the increase in the blood pressure and the increase in the heart rate of each of the nociception data segments;
labeling the prior time period of each of the nociception data segments as prediction data segments;
performing waveform analysis of the prediction data segments to calculate a plurality of signal measures of the prediction data segments; and
determining the prediction input features by computing combinatorial measures between the plurality of signal measures of the prediction data segments and selecting signal measures from the plurality of signal measures of the prediction data segments with most predictive combinatorial measures as the prediction input features.

5. The hemodynamic monitor of claim 4, wherein the system memory comprises hemodynamic drug detection instructions that, when executed by the system processor, are configured to:
extract hemodynamic drug detection input features from the plurality of signal measures that are indicative of a hemodynamic drug administration event of the patient, wherein the hemodynamic drug detection input features, the prediction input features, and the detection input features are extracted concurrently from the plurality of signal measures;
determine a hemodynamic drug detection score of the patient based on based on the hemodynamic drug detection input features, wherein the hemodynamic drug detection score, the nociception prediction score, and the nociception score are determined concurrently;
generate a third sensory alarm signal configured to generate a third sensory alert that indicates that the patient is experiencing the hemodynamic drug administration event when the hemodynamic drug detection score satisfies a predetermined hemodynamic detection criterion;
transmit the third sensory alarm signal to the user interface; and
output the third sensory alert through the user interface.

6. The hemodynamic monitor of claim 5, wherein the hemodynamic drug detection input features of the hemodynamic drug detection instructions are determined by hemodynamic drug detection machine training, wherein the hemodynamic drug detection machine training comprises:
identifying hemodynamic drug administration data segments in the clinical dataset, wherein the hemodynamic drug administration data segments each comprise:
an infusion of a compound that alters cardiovascular hemodynamics;
an increase in blood pressure of at least a third threshold after the infusion; and
an increase in heart rate of at least a fourth threshold after the infusion;
identifying a start and an end of the increase in the blood pressure and the increase in the heart rate in each of the hemodynamic drug administration data segments;
labeling the hemodynamic drug administration data segments after the start and during the increase in the blood pressure and the increase in the heart rate;
performing waveform analysis of the labeled hemodynamic drug administration data segments to calculate a plurality of signal measures of the hemodynamic drug administration data segments; and
determining the hemodynamic drug detection input features by computing combinatorial measures between the plurality of signal measures of the hemodynamic drug administration data segments and selecting signal measures from the plurality of signal measures of the hemodynamic drug administration data segments with most predictive combinatorial measures as being the hemodynamic drug detection input features.

7. The hemodynamic monitor of claim 6, wherein the system memory stores hemodynamic drug prediction instructions that, when executed by the system processor, are configured to:
extract hemodynamic drug prediction input features from the plurality of signal measures that are predictive of a future hemodynamic drug administration event of the patient, wherein the hemodynamic drug prediction input features, the hemodynamic drug detection input features, the prediction input features, and the detection input features are extracted concurrently from the plurality of signal measures;
determine a hemodynamic drug prediction score based on the hemodynamic drug prediction input features, wherein the hemodynamic drug prediction score, the hemodynamic drug detection score, the nociception prediction score, and the nociception score are determined concurrently;
generate a fourth sensory alarm signal configured to generate a fourth sensory alert that indicates that the patient is likely to experience the future hemodynamic drug administration event when the hemodynamic drug prediction score satisfies a predetermined hemodynamic drug prediction criterion;
transmit the fourth sensory alarm signal to the user interface; and
output the fourth sensory alert through the user interface.

8. The hemodynamic monitor of claim 7, wherein the hemodynamic drug prediction input features of the hemodynamic drug prediction instructions are determined by hemodynamic drug prediction machine training, wherein the hemodynamic drug prediction machine training comprises:
identifying a previous time period before the start of the increase in the blood pressure and the increase in the heart rate of each of the hemodynamic drug administration data segments;
labeling the previous time period of each of the hemodynamic drug administration data segments as hemodynamic drug prediction data segments;
performing waveform analysis of the hemodynamic drug prediction data segments to calculate a plurality of signal measures of the hemodynamic drug prediction data segments; and
determining the hemodynamic drug prediction input features by computing combinatorial measures between the plurality of signal measures of the hemodynamic drug prediction data segments and selecting signal measures from the plurality of signal measures of the hemodynamic drug prediction data segments with most predictive combinatorial measures as the hemodynamic drug prediction input features.

9. The hemodynamic monitor of claim 8, wherein the system memory stores stable detection instructions that, when executed by the system processor, are configured to:
extract stable detection input features from the plurality of signal measures that are indicative of a stable episode of the patient where the patient is not experiencing a nociception event nor a hemodynamic drug administration event, wherein the stable detection input features, the hemodynamic drug prediction input features, the hemodynamic drug detection input features, the prediction input features, and the detection input features are extracted concurrently from the plurality of signal measures;
determine a stable score based on the stable detection input features, wherein the stable score indicates a probability of the stable episode, and wherein the stable score, the hemodynamic drug prediction score, the hemodynamic drug detection score, the nociception prediction score, and the nociception score are determined concurrently; and
outputting the stable score to a display.

10. The hemodynamic monitor of claim 9, wherein the stable detection input features of the stable detection instructions are determined by stable detection machine training, wherein the stable machine training comprises:
identifying stable data segments in the clinical dataset, wherein the stable data segments each comprise:
stable blood pressure with no increase greater than the first threshold over a set period of time;
stable heart rate with no increase greater than the second threshold over the set period of time; and
no infusion performed of a compound that alters cardiovascular hemodynamics;
identifying a start and an end of the stable blood pressure and the stable heart rate;
labeling the stable data segments from the start and the end of the stable blood pressure and the stable heart rate;
performing waveform analysis of the labeled stable data segments to calculate a plurality of stable signal measures of the stable data segments; and
determining the stable detection input features by computing combinatorial measures between the plurality of stable signal measures and selecting stable signal measures from the plurality of stable signal measures with most predictive combinatorial measures as being the stable detection input features.

11. The hemodynamic monitor of claim 10, wherein performing waveform analysis of the labeled nociception data segments to calculate a plurality of signal measures of the nociception data segments comprises:
identifying individual cardiac cycles in the arterial pressure waveform of the clinical dataset;
identifying a dicrotic notch in each of the individual cardiac cycles;
identifying a systolic rise phase, a systolic decay phase, and a diastolic phase in each of the individual cardiac cycles; and
extracting signal measures from each of the systolic rise phase, the systolic decay phase, and the diastolic phase from each of the individual cardiac cycles.

12. The hemodynamic monitor of claim 11, wherein:
the signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle;
the signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles; and/or
the signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

13. The hemodynamic monitor of claim 12, wherein computing the combinatorial measures between the plurality of signal measures of the nociception data segments comprises:
performing step one by arbitrarily selecting three signal measures from the plurality of signal measures of the nociception data segments;
performing step two by calculating different orders of power for each of the three signal measures to generate powers of the three signal measures;
performing step three by multiplying the powers of the three signal measures together to generate the product of the powers of the three signal measures;
performing step four by performing receiver operating characteristic (ROC) analysis of the product to arrive at a combinatorial measure for the three signal measures; and
repeating steps one, two, three, and four until all of the combinatorial measures have been computed between all of the plurality of signal measures of the nociception data segments.

14. A hemodynamic monitor for detecting nociception of a patient, the hemodynamic monitor comprising:
an arterial blood pressure sensor comprising a housing, a fluid input port connected via tubing to a fluid source, a catheter-side fluid port connected to a catheter for insertion within an arterial system of the patient, a pressure transducer in communication with the fluid source through the fluid port, and an I/O cable in electrical communication with the pressure transducer; and
an integrated hardware unit comprising:
a system processor;
a system memory;
a display comprising a user interface; and
an analog-to-digital (ADC) converter;
wherein the system memory comprises instructions that, when executed by the system processor, are configured to:
receive an electrical signal from the pressure transducer over a period of time, the electrical signal based on a pressure of the arterial system of the patient transmitted through the fluid source;
convert the electrical signal to a digital signal;
generate an arterial pressure waveform data of the patient based on the digital signal;
extract a plurality of signal measures from the arterial pressure waveform data of the patient;
extract detection input features from the plurality of signal measures that are indicative of a nociception event of the patient;
determine a nociception score of the patient based on the detection input features;
generate a sensory alarm signal configured to generate a sensory alert that indicates that the patient is experiencing a current nociception event when the nociception score satisfies a predetermined detection criterion;
transmit the sensory alarm signal to the user interface; and
output the sensory alert through the user interface.

15. The hemodynamic monitor of claim 14, wherein the detection input features of the nociception detection instructions are determined by detection machine training, wherein the detection machine training comprises:
collecting a clinical dataset containing arterial pressure waveforms and clinical annotations of administrations of a compound that alters cardiovascular hemodynamics;
identifying nociception data segments in the clinical dataset, wherein the nociception data segments each comprise:
an increase in blood pressure of at least a first threshold compared to a prior time period;
an increase in heart rate of at least a second threshold compared to the prior time period; and
no infusion of the compound that alters cardiovascular hemodynamics started prior to the increase in blood pressure and the increase in heart rate;
identifying a start and an end of the increase in the blood pressure and the increase in the heart rate;
labeling the nociception data segments after the start and during the increase in the blood pressure and the increase in the heart rate;
performing waveform analysis of the labeled nociception data segments to calculate a plurality of signal measures of the nociception data segments; and
determining the detection input features by computing combinatorial measures between the plurality of signal measures of the nociception data segments and selecting top signal measures from the plurality of signal measures of the nociception data segments with most predictive combinatorial measures and labeling the top signal measures as the detection input features.

16. The hemodynamic monitor of claim 15, wherein the system memory comprises nociception prediction instructions that, when executed by the system processor, are configured to:
extract prediction input features from the plurality of signal measures that are predictive of a future nociception event of the patient, wherein the prediction input features and the detection input features are extracted concurrently from the plurality of signal measures;
determine a nociception prediction score of the patient based on the prediction input features, wherein the nociception prediction score and the nociception score are determined concurrently;
generate a second sensory alarm signal configured to generate a second sensory alert that indicates that the patient is likely to experience the future nociception event when the nociception prediction score satisfies a predetermined prediction criterion;
transmit the second sensory alarm signal to the user interface; and
output the second sensory alert through the user interface.

17. The hemodynamic monitor of claim 16, wherein the prediction input features of the nociception prediction instructions are determined by prediction machine training, wherein the prediction machine training comprises:
identifying the prior time period before the start of the increase in the blood pressure and the increase in the heart rate of each of the nociception data segments;
labeling the prior time period of each of the nociception data segments as prediction data segments;
performing waveform analysis of the prediction data segments to calculate a plurality of signal measures of the prediction data segments; and
determining the prediction input features by computing combinatorial measures between the plurality of signal measures of the prediction data segments and selecting signal measures from the plurality of signal measures of the prediction data segments with most predictive combinatorial measures as the prediction input features.

18. The hemodynamic monitor of claim 17, wherein the system memory comprises hemodynamic drug detection instructions that, when executed by the system processor, are configured to:
extract hemodynamic drug detection input features from the plurality of signal measures that are indicative of a hemodynamic drug administration event of the patient, wherein the hemodynamic drug detection input features, the prediction input features, and the detection input features are extracted concurrently from the plurality of signal measures;
determine a hemodynamic drug detection score of the patient based on based on the hemodynamic drug detection input features, wherein the hemodynamic drug detection score, the nociception prediction score, and the nociception score are determined concurrently;
generate a third sensory alarm signal configured to generate a third sensory alert that indicates that the patient is experiencing the hemodynamic drug administration event when the hemodynamic drug detection score satisfies a predetermined hemodynamic detection criterion;
transmit the third sensory alarm signal to the user interface; and
output the third sensory alert through the user interface.

19. The hemodynamic monitor of claim 18, wherein the hemodynamic drug detection input features of the hemodynamic drug detection instructions are determined by hemodynamic drug detection machine training, wherein the hemodynamic drug detection machine training comprises:
identifying hemodynamic drug administration data segments in the clinical dataset, wherein the hemodynamic drug administration data segments each comprise:
an infusion of a compound that alters cardiovascular hemodynamics;
an increase in blood pressure of at least a third threshold after the infusion; and
an increase in heart rate of at least a fourth threshold after the infusion;
identifying a start and an end of the increase in the blood pressure and the increase in the heart rate in each of the hemodynamic drug administration data segments;
labeling the hemodynamic drug administration data segments after the start and during the increase in the blood pressure and the increase in the heart rate;
performing waveform analysis of the labeled hemodynamic drug administration data segments to calculate a plurality of signal measures of the hemodynamic drug administration data segments; and
determining the hemodynamic drug detection input features by computing combinatorial measures between the plurality of signal measures of the hemodynamic drug administration data segments and selecting signal measures from the plurality of signal measures of the hemodynamic drug administration data segments with most predictive combinatorial measures as being the hemodynamic drug detection input features.

20. The hemodynamic monitor of claim 19, wherein the system memory stores hemodynamic drug prediction instructions that, when executed by the system processor, are configured to:
extract hemodynamic drug prediction input features from the plurality of signal measures that are predictive of a future hemodynamic drug administration event of the patient, wherein the hemodynamic drug prediction input features, the hemodynamic drug detection input features, the prediction input features, and the detection input features are extracted concurrently from the plurality of signal measures;
determine a hemodynamic drug prediction score based on the hemodynamic drug prediction input features, wherein the hemodynamic drug prediction score, the hemodynamic drug detection score, the nociception prediction score, and the nociception score are determined concurrently;
generate a fourth sensory alarm signal configured to generate a fourth sensory alert that indicates that the patient is likely to experience the future hemodynamic drug administration event when the hemodynamic drug prediction score satisfies a predetermined hemodynamic drug prediction criterion;
transmit the fourth sensory alarm signal to the user interface; and
output the fourth sensory alert through the user interface.

21. The hemodynamic monitor of claim 20, wherein the hemodynamic drug prediction input features of the hemodynamic drug prediction instructions are determined by hemodynamic drug prediction machine training, wherein the hemodynamic drug prediction machine training comprises:
identifying a previous time period before the start of the increase in the blood pressure and the increase in the heart rate of each of the hemodynamic drug administration data segments;
labeling the previous time period of each of the hemodynamic drug administration data segments as hemodynamic drug prediction data segments;
performing waveform analysis of the hemodynamic drug prediction data segments to calculate a plurality of signal measures of the hemodynamic drug prediction data segments; and
determining the hemodynamic drug prediction input features by computing combinatorial measures between the plurality of signal measures of the hemodynamic drug prediction data segments and selecting signal measures from the plurality of signal measures of the hemodynamic drug prediction data segments with most predictive combinatorial measures as the hemodynamic drug prediction input features.

22. The hemodynamic monitor of claim 21, wherein the system memory stores stable detection instructions that, when executed by the system processor, are configured to:
extract stable detection input features from the plurality of signal measures that are indicative of a stable episode of the patient where the patient is not experiencing a nociception event nor a hemodynamic drug administration event, wherein the stable detection input features, the hemodynamic drug prediction input features, the hemodynamic drug detection input features, the prediction input features, and the detection input features are extracted concurrently from the plurality of signal measures;
determine a stable score based on the stable detection input features, wherein the stable score indicates a probability of the stable episode, and wherein the stable score, the hemodynamic drug prediction score, the hemodynamic drug detection score, the nociception prediction score, and the nociception score are determined concurrently; and
outputting the stable score to a display.

23. The hemodynamic monitor of claim 22, wherein the stable detection input features of the stable detection instructions are determined by stable detection machine training, wherein the stable machine training comprises:
identifying stable data segments in the clinical dataset, wherein the stable data segments each comprise:
stable blood pressure with no increase greater than the first threshold over a set period of time;
stable heart rate with no increase greater than the second threshold over the set period of time; and
no infusion performed of a compound that alters cardiovascular hemodynamics;
identifying a start and an end of the stable blood pressure and the stable heart rate;
labeling the stable data segments from the start and the end of the stable blood pressure and the stable heart rate;
performing waveform analysis of the labeled stable data segments to calculate a plurality of stable signal measures of the stable data segments; and
determining the stable detection input features by computing combinatorial measures between the plurality of stable signal measures and selecting stable signal measures from the plurality of stable signal measures with most predictive combinatorial measures as being the stable detection input features.

24. The hemodynamic monitor of claim 23, wherein performing waveform analysis of the labeled nociception data segments to calculate a plurality of signal measures of the nociception data segments comprises:
identifying individual cardiac cycles in the arterial pressure waveform of the clinical dataset;
identifying a dicrotic notch in each of the individual cardiac cycles;
identifying a systolic rise phase, a systolic decay phase, and a diastolic phase in each of the individual cardiac cycles; and
extracting signal measures from each of the systolic rise phase, the systolic decay phase, and the diastolic phase from each of the individual cardiac cycles.

25. The hemodynamic monitor of claim 24, wherein:
the signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle;
the signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles; and/or
the signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

26. The hemodynamic monitor of claim 25, wherein computing the combinatorial measures between the plurality of signal measures of the nociception data segments comprises:
performing step one by arbitrarily selecting three signal measures from the plurality of signal measures of the nociception data segments;
performing step two by calculating different orders of power for each of the three signal measures to generate powers of the three signal measures;
performing step three by multiplying the powers of the three signal measures together to generate the product of the powers of the three signal measures;
performing step four by performing receiver operating characteristic (ROC) analysis of the product to arrive at a combinatorial measure for the three signal measures; and
repeating steps one, two, three, and four until all of the combinatorial measures have been computed between all of the plurality of signal measures of the nociception data segments.

27. A method for monitoring of arterial pressure of a patient for current or predicted future nociception of the patient, the method comprising:
receiving, by a hemodynamic monitor, a continuous signal from a blood pressure sensor connected to the patient over a period of time;
generating, by the hemodynamic monitor, continuously over the period of time an arterial pressure waveform of the patient based on the signal;
extracting, by the hemodynamic monitor, a plurality of signal measures from the arterial pressure waveform of the patient;
extracting, by the hemodynamic monitor input features from the plurality of signal measures that are indicative of a current nociception event of the patient and predictive of a future nociception event of the patient;
determining, by the hemodynamic monitor based on the input features, a nociception score representing a probability of the current nociception event of the patient and/or a probability of the future nociception event of the patient;
invoking, by the hemodynamic monitor, a sensory alarm to produce a sensory signal in response to the nociception score satisfying a predetermined level criterion; and
administering an analgesic to the patient when the nociception score satisfies the predetermined level criterion.

28. The method of claim 27, and further comprising:
determining, by the hemodynamic monitor based on the input features, a hemodynamic drug score representing a probability of a hemodynamic drug administration event of the patient and/or a probability of a future hemodynamic drug administration event of the patient, wherein a hemodynamic drug administration event is defined as the patient experiencing an increase in heart rate and an increase in blood pressure due to administration of a compound that alters cardiovascular hemodynamics and is not an nociception event of the patient, and wherein the hemodynamic monitor determines the hemodynamic drug score and the nociception score concurrently;
determining, by the hemodynamic monitor based on the input features, a stable score representing a probability of a stable episode where the patient is not experiencing a nociception event nor a hemodynamic drug administration event and wherein the hemodynamic monitor determines the stable score and the nociception score concurrently; and
outputting, by the hemodynamic monitor, the stable score, the hemodynamic drug score, and the nociception score concurrently to a display of the hemodynamic monitor.

29. The method of claim 28, and further comprising:
training the hemodynamic monitor for determining the probability of the current nociception event of the patient, wherein the training the hemodynamic monitor comprises:
collecting a clinical dataset including arterial pressure waveforms and clinical annotations of administration of a compound that alters cardiovascular hemodynamics;
identifying nociception data segments in the clinical dataset, wherein the nociception data segments each comprise:
an increase in blood pressure of at least a first threshold compared to a prior time period;
an increase in heart rate of at least a second threshold compared to the prior time period; and
no infusion of the compound that alters cardiovascular hemodynamics started prior to the increase in blood pressure and the increase in heart rate;
identifying a start and an end of the increase in the blood pressure and the increase in the heart rate;
labeling the nociception data segments after the start and during the increase in the blood pressure and the increase in the heart rate;
performing waveform analysis of the labeled nociception data segments to calculate a plurality of signal measures of the nociception data segments; and
determining at least a portion of the input features by computing combinatorial measures between the plurality of signal measures and selecting signal measures from the plurality of signal measures with most predictive combinatorial measures as belonging to the input features.

30. The method of claim 29, and further comprising training the hemodynamic monitor for determining the probability of the predicted future nociception event of the patient by:
identifying the prior time period before the start of the increase in the blood pressure and the increase in the heart rate of each of the nociception data segments;
labeling the prior time period of each of the nociception data segments as prediction data segments;
performing waveform analysis of the prediction data segments to calculate a plurality of signal measures of the prediction data segments; and
determining at least a portion of the input features by computing combinatorial measures between the plurality of signal measures of the prediction data segments and signal measures from the plurality of signal measures of the prediction data segments with most predictive combinatorial measures as belonging to the input features.

31. The method of claim 30, and further comprising:
training the hemodynamic monitor for determining the probability of the hemodynamic drug administration event of the patient, wherein the training the hemodynamic monitor for determining the probability of the hemodynamic drug administration event of the patient comprises:
identifying hemodynamic drug administration data segments in the clinical dataset, wherein the hemodynamic drug administration data segments each comprise:
an infusion of a compound that alters cardiovascular hemodynamics;
an increase in blood pressure of at least a third threshold after the infusion; and
an increase in heart rate of at least a fourth threshold after the infusion;
identifying a start and an end of the increase in the blood pressure and the increase in the heart rate in each of the hemodynamic drug administration data segments;
labeling the hemodynamic drug administration data segments after the start and during the increase in the blood pressure and the increase in the heart rate;
performing waveform analysis of the labeled hemodynamic drug administration data segments to calculate a plurality of signal measures of the hemodynamic drug administration data segments; and
determining at least a portion of the input features by computing combinatorial measures between the plurality of signal measures of the hemodynamic drug administration data segments and selecting signal measures from the plurality of signal measures of the hemodynamic drug administration data segments with most predictive combinatorial measures as belonging to the input features.

32. The method of claim 31, and further comprising:
training the hemodynamic monitor for determining the probability of the future hemodynamic drug administration event of the patient, wherein the training the hemodynamic monitor for determining the probability of the future hemodynamic drug administration event of the patient comprises:
identifying a previous time period before the start of the increase in the blood pressure and the increase in the heart rate of each of the hemodynamic drug administration data segments;
labeling the previous time period of each of the hemodynamic drug administration data segments as hemodynamic drug prediction data segments;
performing waveform analysis of the hemodynamic drug prediction data segments to calculate a plurality of signal measures of the hemodynamic drug prediction data segments; and
determining at least a portion of the input features by computing combinatorial measures between the plurality of signal measures of the hemodynamic drug prediction data segments and selecting signal measures from the plurality of signal measures of the hemodynamic drug prediction data segments with most predictive combinatorial measures as belonging to the input features.

33. The method of claim 32, and further comprising:
training the hemodynamic monitor for determining the probability of the stable episode of the patient, wherein the training the hemodynamic monitor for determining the probability of the stable episode comprises:
identifying stable data segments in the clinical dataset, wherein the stable data segments each comprise:
stable blood pressure with no increase greater than the first threshold over a set period of time;
stable heart rate with no increase greater than the second threshold over the set period of time; and
no infusion performed of a compound that alters cardiovascular hemodynamics;
identifying a start and an end of the stable blood pressure and the stable heart rate;
labeling the stable data segments from the start and the end of the stable blood pressure and the stable heart rate;
performing waveform analysis of the labeled stable data segments to calculate a plurality of stable signal measures of the stable data segments; and
determining at least a portion of the input features by computing combinatorial measures between the plurality of stable signal measures and selecting stable signal measures from the plurality of stable signal measures with most predictive combinatorial measures as belonging to the input features.
